(19) United States
(12) Patent Application Publication
(10) Pub. No.: US 2008/0249543 A1
(43) Pub. Date: Oct. 9, 2008

(54) Ligature Carrier

(76) Inventor: George Kaladelfos, New South Wales (AU)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/580,756

(22) PCT Filed: Nov. 26, 2004
(86) PCT No.: PCT/AU04/01674

§ 371 Date: Apr. 10, 2007

(30) Foreign Application Priority Data
Nov. 27, 2003 (AU) 2003906592

(51) Int. Cl. A61B 17/04; A61B 17/10

(52) U.S. Cl. 606/144; 606/139

(57) ABSTRACT

A ligament carrier and suturing device (20) comprising an elongate shaft member having a distal end and a proximal end. A needle carrier (40) is located at or adjacent the distal end of said elongate shaft member, said needle carrier comprising a rigid arcuate tooth member and having a first end and a second end and being pivotable at the first end about an axis which lies transverse to the longitudinal axis of the shaft member (30), the needle carrier adapted to carry a needle (80) with a suture (90) attached thereto. A needle capture means (100) is located on the shaft member, said needle capture device being adapted to engage with and capture needle carried by arcuate tooth. An operating means (50) coupled to the needle carrier and operable to cause the arcuate tooth member to pivot through an arc so that the needle engages with and is captured by the capture device, the operating means being operable from a location at or adjacent said proximal end of the shaft member.

### FIELD OF THE INVENTION

The present invention relates to a surgical device of the type which can be used for inserting sutures within a human or animal body through a confined opening. The invention will be particularly described with reference to a ligature carrier particularly suited for transvaginal sacrospinous colpopexy, but it is to be understood that the device of the invention may be used in other applications.

### BACKGROUND OF THE INVENTION

Transvaginal colpopexy has become, in recent years, the preferred treatment of vaginal or vault prolapse or vaginal eversion. This condition tends to be caused by failure of the supporting mechanism of the upper genital tract. Treatment of the condition requires the attachment of the vault to a suitable attachment point within the body, the preferred attachment points being the coccygeus muscle-sacrospinal ligament complex located on either side of the patient's pelvis. Attachment of each side of the vault to the left and right sacrospinal ligaments has been performed either abdominally or transvaginally, the latter procedure being preferred for reasons such as patient health and wellbeing.

One problem with performing the procedure transvaginally is that the surgeon is obliged to perform the procedure through the relatively confined vaginal passage. Furthermore, the artificial ligaments need to be attached at some depth within the patient's body, typically the sacrospinal ligaments, which has meant that some form of elongate ligature carrier and suturing device is required to perform the procedure.

Various prior art elongate suturing devices have been developed, but for one or other reason the prior art devices currently available are not ideal for performing the procedure. One reason for this is that the sacrospinal ligaments run transverse to the length of the vaginal passage. It is desirable for the sutures to be placed transverse to the length of the ligaments, not parallel to the length of the ligaments, but prior art devices make it technically difficult to place the sutures in this way.

It is also desirable that, whatever elongate suturing device is used for placing the sutures in the ligaments, the device can effectively perform the procedure on its own, without requiring the assistance of either the surgeon's finger tips, or another instrument used in conjunction with the suturing device. This is due to the fact that the passage through which the surgeon is working is narrow and if another instrument or the surgeon's other hand occupies the passage then placing the suture has to be done ‘blind” or by feel, and this makes the procedure far more difficult. If the surgeon is obliged to use two instruments in the suturing procedure then both hands are occupied with suturing, rather than one being free to manipulate or orientate the vaginal passage with retractors.

Various instruments have been developed specifically for performing the procedure. These include the Veronikis device, described in Obstetrics & Gynecology, Vol 89, No 3, March 1997. This device, however, requires the surgeon to use a suture hook to capture the suture once it has passed through the ligament. Another device, the Miya hook, requires the surgeon to use his or her fingers of the free hand to properly place the suture. A Laurus needle driver has been used to perform the procedure (see Obstetrics & Gynecology, Vol 89, No 1, January 1997). This procedure requires both the surgeon to use his or her fingers in the placement, as well as retrieving the needle after it has passed through the ligament. A further device which is used to perform the procedure is the Shutt Suture Punch, but once again, this device requires the suture to be retrieved after it has passed through the ligament.

A further problem encountered when performing the procedure is that the coccygeus muscle-sacrospinal ligament complex tends to lie somewhat flat, and therefore does not present well to the suturing device. This is one reason why, with at least some devices, the surgeon needs to use the finger tips of the free hand to press the ligament into a mounded configuration so that the suturing device can take a firm bite of the ligament. Once again, it would be far preferable if the device could function in such a way that the suture could be placed using the device alone, but yet ensure that the suture is passed through sufficient ligament to properly and safely be used to anchor the vault in place.

Any discussion of documents, publications, acts, devices, substances, articles, materials or the like which is included in the present specification has been done so for the sole purpose so as to provide a contextual basis for the present invention. Any such discussions are not to be understood as admission of subject matter which forms the prior art base, or any part of the common general knowledge of the relevant technical field in relation to the technical field of the present invention to which it extended at the priority date or dates of the present invention.

### SUMMARY OF THE INVENTION

According to the invention there is provided a ligament carrier and suturing device comprising:
an elongate shaft member having a distal end and a proximal end;
a needle carrier located at or adjacent the distal end of said elongate shaft member, said needle carrier comprising a rigid arcuate tooth member and having a first end and a second end and being pivotable at the first end about an axis which lies transverse to the longitudinal axis of the shaft member, the needle carrier adapted to carry on the second end thereof a needle with a suture attached thereto;
a needle capture means located on the shaft member intermediate the distal and proximal ends thereof, said needle capture device being adapted to engage with and capture the needle carried by the arcuate tooth; and
an operating means coupled to the needle carrier and operable to cause the arcuate tooth member to pivot through an arc so that the needle engages with and is captured by the capture device, the operating means being operable from a location at or adjacent said proximal end of the shaft member.

Preferably, the operating means comprises a connecting rod located within a longitudinal passage extending along said shaft member and being engaged with said needle carrier. Preferably the engagement between the connecting rod and the needle carrier comprises a lever arrangement adapted to drive the arcuate tooth through said arc.

Preferably the needle carrier includes a slot, said connecting rod being slidingly engaged with said slot so as to cause the needle carrier to pivot about the first end as said connecting rod moves longitudinally in relation to said shaft member. Preferably, the needle carrier is biased such that the needle is urged away from said needle capture means, At least a portion of the second end of the needle carrier may be hollow to receive the needle therein and the second end of the needle carrier may include a slot arranged to receive an axially aligned suture connected to the needle such that the suture passes from inside to outside of the needle carrier.

Preferably a handle means is located at or adjacent the proximal end of said shaft member. The handle means may be coupled to said operating means such that movement of said handle means causes the needle carrier to be advanced towards said needle capture means. The handle means may be arranged such that the device is held and operated by a single hand of a user. Preferably the handle means comprises a moveable lever and a grip, said lever being coupled to said operating means.

Preferably, the needle capture means is biased such that when the needle is advanced by said needle carrier to said needle capture means the needle capture means engages with the needle automatically, and when the needle carrier is moved away from the needle capture means, the needle disengages from said needle carrier and remains captured. Alternatively, the needle capture means may be manually operable by a user from the proximal end of the shaft member. Preferably the needle capture means captures the needle by engaging with a recess in the needle. The needle capture means may be manually operable from the proximal end of the shaft member by a user to allow the needle to be released from the needle capture means.

These and further features of the invention will be made apparent from the description of a preferred embodiment of the invention, given below by way of example. In the description reference is made to the accompanying drawings, but the specific features shown in the drawings should not be construed as limiting on the invention.

Throughout the specification the term “comprise” and variations on this term including “comprising” and “comprises” are to be understood to imply the inclusion of a feature, integer, step or element, and not exclude other features, integers, steps or elements.

### BRIEF DESCRIPTION OF THE DRAWINGS

The invention now will be described, by way of example only, and with reference to the accompanying drawings in which:

FIG. 1 shows diagrammatically the surgical approach which a surgeon may follow for vaginal sacrospinal fixation;

FIG. 2 shows the manner of attachment of the vagina to the sacrospinous ligaments in sacrospinal fixation;

FIG. 3 shows a part sectional view of an embodiment of a suturing device according to the present invention prior to a suture being deployed;

FIG. 4 depicts the suturing device of FIG. 3 during deployment of the suture;

FIG. 5 depicts the suturing device of FIG. 3 and FIG. 5 after deployment of the suture;

FIG. 6 shows an enlarged sectional side view of a first embodiment of a needle and capture device according to the present invention;

FIG. 7 shows an enlarged sectional side view of a second embodiment of a needle and capture device according to the present invention; and

FIG. 8 shows an enlarged sectional side view of a third embodiment of a needle and capture device according to the present invention.

### DETAILED DESCRIPTION OF THE EMBODIMENTS

The following description refers to a preferred embodiment of the present invention whereby the present invention is directed to a surgical device of the type for inserting sutures within a human or animal body through a confined opening. Reference in the preferred embodiment below is made with respect to transvaginal sacrospinous colpopexy.

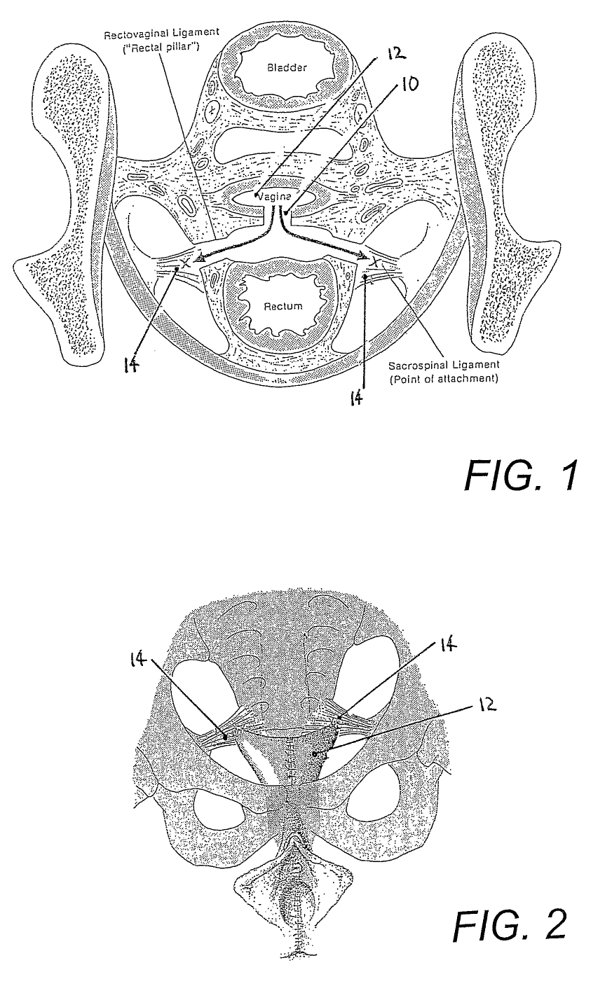

Referring to FIG. 1 and FIG. 2, as discussed above, the operation known as sacrospinous vaginal vault suspension requires a surgeon to attached the rear or inner portion of the vaginal vault, typically on opposite sides of the vault, to the two sacrospinal ligaments 14. As shown, an incision 10 is made through the lower rear wall of the vagina 12 in order to obtain access to the sacrospinal ligaments 14 located on opposite sides of the patient. The attachment points are indicated by the letter X on the ligaments. Once sutures have been located in the ligaments 14, these may be used to connect artificial ties or ligaments, similarly as in the manner described in patent application PCT/AU00/01298. The artificial ties that are used are typically in a form of an inert mesh material adapted to remain permanently within the body of the subject. Further information relating to the manner in which such a procedure is performed may be obtained by reference to the two Obstetrics & Gynaecology articles referred to in the background section of the specification. The information contained in those two documents are incorporated herein by way of reference. Once the operation has been completed the wall of the vagina will be repaired, with the vagina remaining secured to the sacrospinal ligaments 14 using the artificial ties as indicated in FIG. 2 of the drawings.

The present inventor has observed that for the reasons discussed above, inserting the sutures into the sacrospinal ligaments 14 of a patient can often be difficult, resulting in a delay to the completion of the operation or, unless care is taken, inferior attachment to the ligaments. Furthermore, as previously discussed, it is important that the sutures be placed transverse to the length of the ligaments 14, rather than parallel to the length of the ligament 14 in order to achieve optimal and safe attachment.

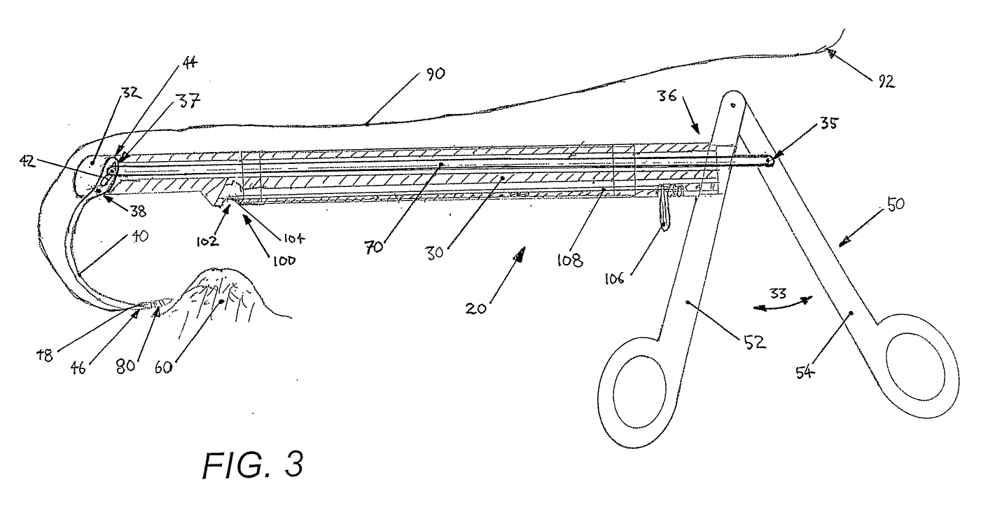

Referring to FIGS. 3, 4 and 5, an embodiment of a device according to the present invention and its manner of use is depicted. It will be appreciated that the device shown is depicted in somewhat schematic fashion but it is evident from the drawings how such a device may be used in practice. Furthermore, although shown diagrammatically, it will be understood that alternate shapes, geometries and sizes of the various features of the invention may be used in other embodiments without departing from the scope of the invention as claimed.

As shown, the suturing device 20 comprises an elongate shaft member 30 having a C-shaped needle carrier 40 located a first end 32 thereof and a handle member 50 located at a second end 36. Clearly the first end 32 is the end which is to be inserted into the patient's vagina, through the incision 10 of FIG. 1, in order to make the attachment X in the ligaments 14. Thus, the length, shape and size of the shaft member 30 is selected to be able to optimally perform the operation. Where it is desired to use the suturing device 20 in other types of operations or procedures, clearly the configuration including the length of the shaft may vary from that shown herein in the present embodiment of the invention. In the present embodiment, when used in transvaginal application, it is envisaged that the overall length of the device will be about 25 cm although, clearly, different length devices could be utilised.

The needle carrier 40 acts as a tooth or spike to pass through the ligament or other tissue through which it is desired to pass a suture. As shown, the tissue is indicated at numeral 60, that being the sacrospinal ligaments referred to above.

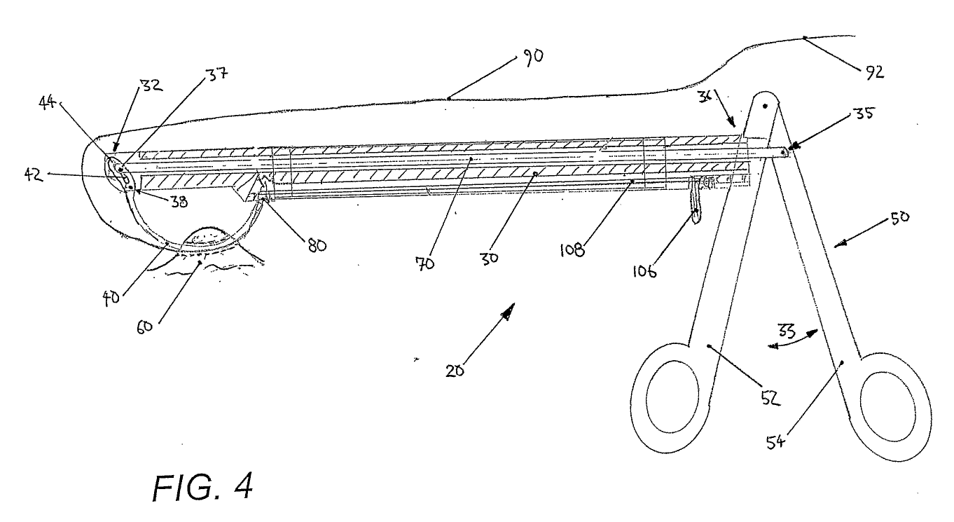

The needle carrier 40 is pivotally connected to the shaft member 30 through pivot pin 38 to extreme positions being distal the end 32 of the shaft 30. Thus, the needle carrier 40 is able to pivot between a retracted position as shown in FIG. 3, and an engaged position shown in FIG. 4.

The handle means 50 is used to move the needle carrier 40 between the two extreme positions. As shown, the handle means 50 has a fixed leg 52 and a pivotable leg 54 which can be moved forwards and backwards in the direction of arrow 33. A connecting rod 70 is connected to the movable leg 54 via a pivotal connection 35 at the second end 36 of the shaft member 30, and at the first end 32 of the shaft member is connected to the needle carrier 24 via pivot pin 37 which locates in a slot 42 formed in the proximal end 44 of the needle carrier 40. Thus, when the rod 36 moves back and forward in relation to the longitudinal axis of the shaft member 30, the pin 37 carries the needle carrier 40 with it, causing the needle carrier to pivot about a pivot pin 38. The connecting rod 70 thus acts on the needle carrier 40 in the manner of a lever to operate the suturing device 20.

The needle carrier 40 carries a short stub needle 80 on the distal end 46 or forward end thereof, the stub needle 80 being a close or sliding fit in the distal end 46 of the needle carrier 40, the distal end 46 being tubular. The stub needle 80 and the distal end 46 of the needle carrier 40 are engaged in a manner such that the stub needle 80 is prevented from inadvertently detaching from the needle carrier 40 in use. However, when gripped, the stub needle 80 detaches from the needle carrier 40, in the manner as described below.

The stub needle 40 is coaxially joined to a suture 90 in the manner as shown in FIGS. 3 to 5, the suture 90 extending rearwardly from the stub needle 80 and out of the tubular distal end portion 46 of the needle carrier 40 through a slot 48 in the needle carrier 40. It is considered advantageous that the suture 90 is coaxially joined to the stub needle 80 such that if, inadvertently, the stub needle 80 becomes detached from the needle carrier 40 prior to the operation being completed, the needle 46 may be withdrawn from the patient by simply pulling on the free end 92 of the suture 90, that is, from outside of the patient.

In order to pass the suture 90 through the ligament 60 the first end 32 of the shaft member 30 of the suturing device 20 is passed into the patient, in the manner as described above, until the stub needle 80 is located at the far side of the ligament 60. This is the position as depicted in FIG. 3 of the drawings. Thereafter the device may be pulled slightly in order to ensure the stub needle 80 is at least slightly embedded in the ligament 60. It will be appreciated that during this operation, the surgeon is able to visually inspect the location of the stub needle 80 as since the shaft member 30 is preferably relatively slender and will not obscure the surgeon's view. Also, the surgeon does not need to place the fingers of his or her other hand within the patient in order to ensure the location of the stub needle 80 is correct. As mentioned above, the ability to place the suture 90 using only one hand and having an unimpeded view is considered to be important from a practical standpoint.

When the appropriate location of the stub needle 80 has been determined and the stub needle 80 appropriately located, the pivotable leg 54 of handle means 50 may be moved towards the fixed leg 52 of the handle means 50, thereby pushing the connecting rod 70 distally which in turn causes the needle carrier 40 to pivot about pin 38 and travel through the ligament 60 into a position as shown in FIG. 4 of the drawings.

The suturing device 20 further includes a needle capture means 100, in this embodiment being formed integrally with and located at the underside of the shaft member 30. The needle capture means 100 includes a receiving aperture 102 extending into the shaft member 30 into which the stub needle 80 is driven as the needle carrier 40 rotates about pivot pin 38. The needle capture means 100 includes a catch mechanism 104 which is arranged so as to engage with the stub needle 80.

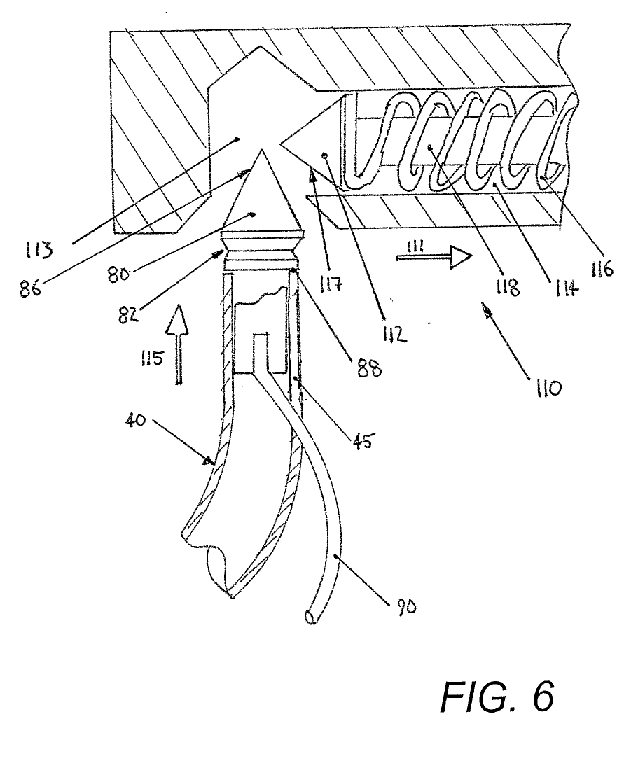

The needle capture means 100 may operate automatically by a spring mechanism biased to as to cause the stub needle 80 to be captured and is discussed in more detail with respect to FIGS. 6 to 8 below. In the present embodiment, the needle capture means 100 includes a lever 106 operably connected to the catch mechanism 104 by a tie rod 108 which allows the catch mechanism 104 to retracted so as to release the stub needle 80.

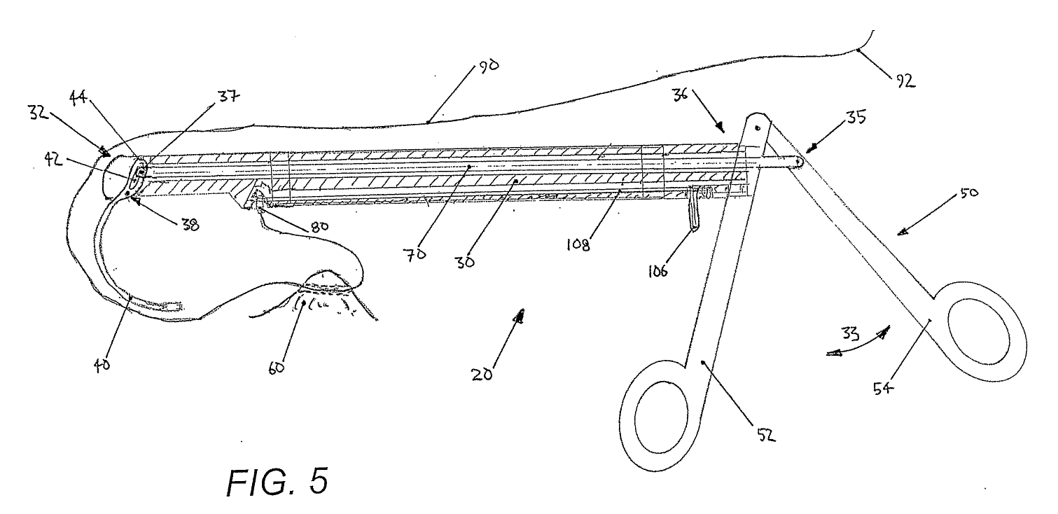

Although in the present embodiment the needle capture means 100 is automatically operated by a spring mechanism, it is envisaged that some surgeons may prefer to manually cause engagement in which case a connection mechanism other than a spring loaded latch mechanism will be used to engage and capture the stub needle 80. Once the stub needle 80 has been engaged, as depicted in FIG. 4, the pivotable leg 54 of the handle is moved in a direction away from the fixed leg 52 causing the needle carrier 40 to be retracted from its fully advanced position towards the position shown in FIG. 5. However, as the stub needle 80 is now captively held in the aperture 102, this movement causes the stub needle 80 to be disengaged from the end 46 of the needle carrier 40 as shown whilst maintaining the stub needle 80 engaged with the needle capture means 100 as the needle carrier 40 is retracted to an advanced position. As the needle carrier 40 is advanced away from the stub needle 80, the suture 90 slides through the slot 48.

By fully retracting the needle carrier 40 to a fully retracted position, the needle carrier 40 will be caused to reverse out of the ligament 60 leaving the suture 90 in position. As the suture 90 is located in the slot 48 in the needle carrier 40, as the stub needle 80 is withdrawn from the needle carrier 40, the suture 90 will disengage from the needle carrier 40 out of the slot 48.

The suturing device 20 device may now be retracted from the patient and by slowly pulling the device 20 whilst the stub needle 80 is retained by the needle capture means 100 in the aperture 102 the suture 90 is pulled through the ligament 60 until the two ends of the suture are located outside of the patient. Once this has been achieved the suture 90 may be severed from the stub needle 80 and the surgeon can proceed with attaching the artificial ligament to the sacrospinal ligament in the manner disclosed in patent application PCT/AU00/01298. Further description of how the operation is completed need not be discussed herein, but it will be understood that by transferring the stub needle 80 from the needle carrier 40 to the aperture 102 and then retracting the needle carrier 80, a one handed insertion of the suture 90 into the ligament 60 can be achieved.

Furthermore, because of the curved configuration of the needle carrier 40 the needle can be driven in a circular arc, and also the hook shaped nature of the needle carrier 40 ensures that a suitable bite of the ligament 60 can be achieved without the surgeon having to manipulate the ligament with his or her fingers. Furthermore, the geometry of the various members of the device 20 and the offset between pivot point of the mechanism may be arranged so as to provide appropriate mechanical advantage for driving the needle carrier 40 through the ligament 60. It is envisaged that the needle carrier 40 will have a radius of curvature of between about 5 mm and 30 mm although a radius of curvature of about 10 mm is considered to be the preferred configuration for this type of operation.

Three different engagement arrangements between the stub needle 80 and the needle capture means 100 are depicted in FIGS. 6 to 8. It must be understood, however, that other arrangements are possible but what is required is a relatively simple arrangement that reliably captures the needle and does not require excess force for engagement.

In FIG. 6, another embodiment of a needle capture means 110 is depicted having a spring loaded latch 112 which is slidable in a slot 114 which lies parallel to the length of the shaft 30 of the device as described above. A compression spring 116 is located around a connecting rod 118 of the latch 112 and the latch 112 will move in the direction of arrow 111 when the stub needle 80 is moved into the aperture 113 in the direction of arrow 115. It will be noted that the latch 112 has a bevelled surface 117 against which the stub needle 80 will press in order to urge the latch back against the action of the spring 116. Once the stub needle 80 has been fully advanced into the aperture 113, the latch 112 will engage in an annular groove 82 located behind the sharp end or head of the stub needle 86. The compression spring 116 will then hold the latch 112 in the groove 82 so that when the needle carrier 40 is retracted, the stub needle 80 remains captured in the aperture 113. It will be noted that the stub needle 80 has a shoulder 88 which will located against the forward end of the needle carrier 80. Also the needle carrier 80 is provided with the slot 45 to allow the suture 90 to connect axially into the rear end of the stub needle 80.

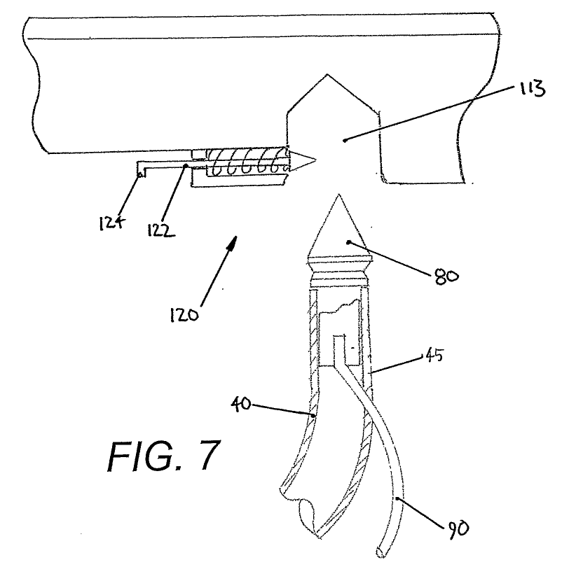

The needle capture means 120 as depicted in FIG. 7 of the drawing is similar to that as depicted in FIG. 6 except that the latch device 122 is provided with a forward facing lever 124. This lever 124 can only be used to disengage the stub needle 80 from the aperture 113 when the device 20 is removed from the patient, but since this is typically the only time when the stub needle 80 would be required to be disengaged from the aperture 113, this arrangement may be preferable in some instances.

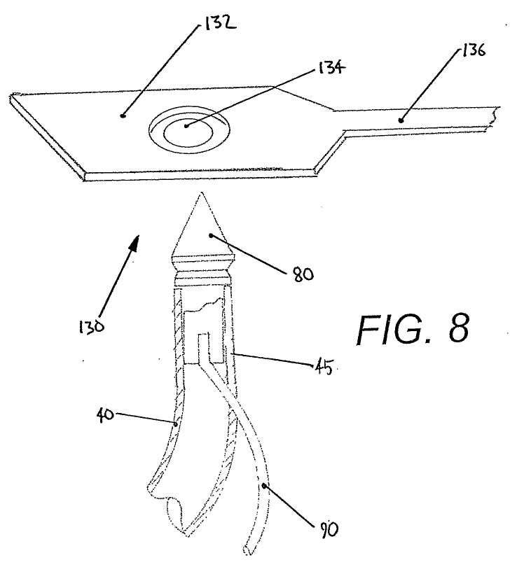

The needle capture means 130 as shown in FIG. 8 provides a narrow plate 132 with a aperture 134 therein through which the stub needle 80 passes as the stub needle passes into the opening 113 as shown in FIGS. 6 and 7. The plate 132 is connected to rod 136 which can either be manually manipulable from the rear end of the suturing device 20, or may be spring loaded to allow for automatic latching of the needle as it is advanced into its fully advanced position, similarly as described with reference to the above embodiments of needle capture means.

Clearly there may be many variations to the above described embodiments without departing from the scope of the invention. Some of the features which are considered to be important are the curved nature of the needle carrier which allows the device to take a “bite” of tissue, such as the ligament described herein, even at relatively remote locations within the body, and even where the tissue to be engaged lies relatively flat. The maimer in which the needle detaches from the needle carrier and the fact that the needle could, if necessary, be retracted from the tissue by pulling on the free end of the suture is also considered advantageous over at least some prior art devices. As mentioned previously, where the device is to be used for suturing different types of operations the overall configuration of the device might need to be quite different from that described herein.

It will be understood that the invention disclosed and defined in this specification extends to all alternative combinations of two or more of the individual features mentioned or evident from the text or drawings. All of these different combinations constitute various alternative aspects of the invention.

1. A ligature carrier and suturing device comprising:
an elongate shaft member having a distal end and a proximal end;
a needle carrier located at or adjacent the distal end of said elongate shaft member, said needle carrier comprising a rigid arcuate tooth member and having a first end and a second end and being pivotable at the first end about an axis which lies transverse to the longitudinal axis of the shaft member, the needle carrier adapted to carry on the second end thereof a needle with a suture attached thereto;
a needle capture means located on the shaft member intermediate the distal and proximal ends thereof, said needle capture device being adapted to engage with and capture the needle carried by the arcuate tooth; and
an operating means coupled to the needle carrier and operable to cause the arcuate tooth member to pivot through an arc so that the needle engages with and is captured by the capture device, the operating means being operable from a location at or adjacent said proximal end of the shaft member.

2. A ligature carrier and suturing device according to claim 1, wherein the operating means comprising a connecting rod located within a longitudinal passage extending along said shaft member and being engaged with said needle carrier.

3. A ligature carrier and suturing device according to claim 2, wherein the engagement between the connecting rod and the needle carrier comprises a lever arrangement adapted to drive the arcuate tooth through said arc.

4. A ligature carrier and suturing device according to claim 2, wherein the needle carrier includes a slot, said connecting rod being slidingly engaged with said slot so as to cause the needle carrier to pivot about the first end as said connecting rod moves longitudinally in relation to said shaft member.

5. A ligature carrier and suturing device according to claim 1, wherein the needle carrier is biased such that the needle is urged away from said needle capture means.

6. A ligature carrier and suturing device according to claim 1, wherein at least a portion of the second end of the needle carrier is hollow to receive the needle therein.

7. A ligature carrier and suturing device according to claim 6, wherein the second end of the needle carrier includes a slot arranged to receive an axially aligned suture connected to the needle such that the suture passes from inside to outside of the needle carrier.

8. A ligature carrier and suturing device according to claim 1 further comprising a handle means located at or adjacent the proximal end of said shaft member.

9. A ligature carrier and suturing device according to claim 8, wherein the handle means is coupled to said operating means such that movement of said handle means causes the needle carrier to be advanced towards said needle capture means.

10. A ligature carrier and suturing device according to claim 9, wherein said handle means is arranged such that the device is held and operated by a single hand of a user.

11. A ligature carrier and suturing device according to claim 9, wherein said handle means comprises a moveable lever and a grip, said lever being coupled to said operating means.

12. A ligature carrier and suturing device according to claim 1, wherein the needle capture means is biased such that when the needle is advanced by said needle carrier to said needle capture means the needle capture means engages with the needle automatically, and when the needle carrier is moved away from the needle capture means, the needle disengages from said needle carrier and remains captured.

13. A ligature and suturing device according to claim 1, wherein the needle capture means is manually operable by a user from the proximal end of the shaft member.

14. A ligature and suturing device according to claim 12, wherein the needle capture means captures the needle by engaging with a recess in the needle.

15. A ligature and suturing device according to claim 12, wherein the needle capture means is manually operable from the proximal end of the shaft member by a user to allow the needle to be released from the needle capture means.

16. (canceled)

17. A ligature and suturing device according to claim 13, wherein the needle capture means captures the needle by engaging with a recess in the needle.

18. A ligature and suturing device according to claim 13, wherein the needle capture means is manually operable from the proximal end of the shaft member by a user to allow the needle to be released from the needle capture means.
